(19) United States
(12) Patent Application Publication
(10) Pub. No.: US 2022/0033833 A1
(43) Pub. Date: Feb. 3, 2022

(54) COMPOSITIONS AND METHODS FOR TRANSFERRING BIOMOLECULES TO WOUNDED CELLS

(71) Applicant: Monsanto Technology LLC, St. Louis, MO (US)

(72) Inventors: Larry A. Gilbertson, Chesterfield, MO (US); Andrei Y. Kouranov, Chesterfield, MO (US); Vladimir A. Sidorov, Chesterfield, MO (US)

(21) Appl. No.: 17/276,455

(22) PCT Filed: Oct. 1, 2019
(86) PCT No.: PCT/US2019/053956

§ 371 Date: Mar. 15, 2021

Related U.S. Application Data
(60) Provisional application No. 62/740,144, filed on Oct. 2, 2018.

(51) Int. Cl. C12N 15/82; C12N 15/11; C12N 9/22

(52) U.S. Cl. CPC C12N 15/8213; C12N 15/8206; C12N 2800/80; C12N 9/22; C12N 2310/20; C12N 15/11

(57) ABSTRACT

The invention provides novel methods and compositions for introduction, transfer or delivery of one or more biomolecules into wounded recipient plant cell(s). Methods for production of a wounded recipient cell culture and the creation of one or more mutations, edits, transgenic insertions, or other genetic changes in the recipient cell(s) are also provided. Product cells produced by such methods, and resulting cells and regenerated plants, plant parts, and progeny plants are further provided. Molecular and genetic analyses, analysis of phenotypes and traits, and use of screenable and selection markers, are also provided to confirm transfer of the biomolecule in to the recipient cell(s) and generation of the mutation, edit, transgenic insertion, or other genetic change in the recipient cell(s), and/or progeny thereof, and in plants or plant parts developed or regenerated from the foregoing.

### CROSS-REFERENCE TO RELATED APPLICATIONS

This application claims the benefit of priority of U.S. Provisional Appl. Ser. No. 62/740,144, filed Oct. 2, 2018, the disclosure of which is hereby incorporated by reference in its entirety.

### FIELD OF THE INVENTION

The invention relates generally to the fields of agriculture, plant biotechnology, and molecular biology. More specifically, the invention relates to compositions and methods for mutating, editing or genetically modifying plant cells.

### BACKGROUND

The ability to create plants having novel combinations of genetic traits is useful for improving crop yields and resisting disease and pest pressures. In addition to crossing or breeding plants together, novel combinations of traits can be introduced transgenically or through various mutagenesis techniques. However, many plant species and varieties are difficult to transform, culture and/or regenerate from an explant or plant material. A need exists in the art for novel and improved methods for transferring genetic elements and molecular tools to regenerable plant cells to create desired traits.

### SUMMARY

In one aspect, the present invention provides a method for transfer of a biomolecule into a cell comprising: a) mixing a recipient plant cell culture comprising at least one recipient cell with a medium comprising at least one biomolecule to obtain a mixed cell culture comprising the recipient cell; and b) wounding the recipient cell of the mixed cell culture to produce at least one product cell into which transfer of the biomolecule has occurred following said mixing and/or wounding. In some embodiments, the recipient plant cell culture, medium or mixed cell culture further comprises an osmoticum. In further embodiments, the osmoticum comprises polyethylene glycol (PEG). In yet further embodiments, the osmoticum comprises a sugar or sugar alcohol. In some embodiments, one or more recipient cells of the recipient plant cell culture comprise a genotype, genetic background, transgene, native allele, edit or mutation of interest. In other embodiments, the at least one product cell, or a progeny cell thereof, comprises the genotype, genetic background, transgene, native allele, edit or mutation of interest from the recipient plant cells. In some embodiments, the recipient plant cell culture is a callus culture or cell suspension culture. In some embodiments, the at least one biomolecule comprises a site-specific nuclease, a guide RNA, or one or more recombinant DNA molecules comprising a sequence encoding a site-specific nuclease and/or a sequence encoding a guide RNA. In further embodiments, the site-specific nuclease is a zinc-finger nuclease (ZFN), a meganuclease, an RNA-guided endonuclease, a TALE-endonuclease (TALEN), a recombinase, or a transposase. In some embodiments, cells of the recipient plant cell culture are dicot plant cells. In further embodiments, the dicot plant cells are selected from the group consisting of tobacco, tomato, soybean, canola, and cotton cells. In other embodiments, cells of the recipient plant cell culture are monocot plant cells. In further embodiments, the monocot plant cells are selected from the group consisting of corn, rice, wheat, barley, and sorghum cells. The present invention further provides a product cell produced by the method described herein. In some embodiments, the product cell is a dicot plant cell. In further embodiments, the product cell is selected from the group consisting of: a tobacco, a tomato, a soybean, a canola, and a cotton plant cell. In other embodiments, the product cell is a monocot plant cell. In further embodiments, the product cell is selected from the group consisting of: a corn, a rice, a wheat, and a sorghum plant cell. A plant regenerated from the product cell produced by the method provided herein, or a progeny cell thereof is also provided. In some embodiments, the regenerated plant is a dicot plant. In further embodiments, the dicot plant is selected from the group consisting of: a tobacco, a tomato, a soybean, a canola, and a cotton plant. In other embodiments, the regenerated plant is a monocot plant. In further embodiments, the monocot plant is selected from the group consisting of: a corn, a rice, a wheat, a barley, and a sorghum plant. Seed, progeny plants, or progeny seed of the regenerated monocot and dicot plants are also provided herein. Also provided herein is a wounded mixed cell culture produced by the method described herein.

In another aspect, the present invention provides a method for transfer of a biomolecule into a cell comprising: a) mixing a recipient plant cell culture comprising at least one recipient cell with a medium comprising at least one biomolecule to obtain a mixed cell culture comprising the recipient cell; and b) wounding the recipient cell of the mixed cell culture to produce at least one product cell into which transfer of the biomolecule has occurred following said mixing and/or wounding, wherein the recipient plant cell culture comprises cells having a plastid genome-encoded marker gene and/or a nuclear genome-encoded marker gene. In some embodiments, the method further comprises the step of: c) screening or selecting for the at least one product cell of the mixed cell culture, or at least one progeny cell thereof, or a plant developed or regenerated from the at least one product cell, or a progeny cell thereof, based on the presence of the nuclear genome encoded marker gene and/or plastid genome-encoded marker gene, during and/or after step (b). In other embodiments, the nuclear genome encoded marker gene or the plastid genome-encoded marker gene is a selectable marker gene. In further embodiments, the selectable marker gene is selected from the group consisting of: aadA, rrnS, rrnL, nptII, aphA-6, psbA, bar, HPPD, ASA2, and AHAS. In some embodiments, the nuclear genome encoded marker gene or the plastid genome-encoded marker gene is a screenable marker gene. In further embodiments, the screenable marker gene is gfp or gus. In some embodiments, the cells of the recipient plant cell culture, or progeny cells thereof, are homoplastomic for the plastid-encoded marker gene.

In another aspect, the present invention provides a method for transfer of a biomolecule into a cell comprising: a) mixing a recipient plant cell culture comprising at least one recipient cell with a medium comprising at least one biomolecule to obtain a mixed cell culture comprising the recipient cell; b) wounding the recipient cell of the mixed cell culture to produce at least one product cell into which transfer of the biomolecule has occurred following said mixing and/or wounding; and c) screening or selecting for the at least one product cell, or a progeny cell thereof, or a plant developed or regenerated from the at least one product cell, or a progeny cell thereof, based on a selectable or screenable marker. In some embodiments, the method further comprises the step of: d) regenerating a plant from the mixed cell culture and/or the at least one product cell, or at least one progeny cell thereof.

In yet another aspect, the present invention provides a method for transfer of a biomolecule into a cell comprising: a) mixing a recipient plant cell culture comprising at least one recipient cell with a medium comprising at least one biomolecule to obtain a mixed cell culture comprising the recipient cell; b) wounding the recipient cell of the mixed cell culture to produce at least one product cell into which transfer of the biomolecule has occurred following said mixing and/or wounding; and c) adding an osmoticum to the recipient plant cell culture, medium or mixed cell culture prior to, during or after step a) or step b). In some embodiments, the osmoticum comprises polyethylene glycol (PEG). In other embodiments, the osmoticum comprises a sugar or sugar alcohol.

In yet another aspect, the present invention provides a method for transfer of a biomolecule into a cell comprising: a) mixing a recipient plant cell culture comprising at least one recipient cell with a medium comprising at least one biomolecule to obtain a mixed cell culture comprising the recipient cell; b) wounding the recipient cell of the mixed cell culture to produce at least one product cell into which transfer of the biomolecule has occurred following said mixing and/or wounding; and c) screening or selecting for at least one edited or mutated product cell, or a progeny cell thereof, or a plant developed or regenerated from the at least one edited product cell, or a progeny cell thereof, having the edit or mutation. In some embodiments, the plant developed or regenerated from the at least one edited or mutated product cell, or a progeny cell thereof, is screened or selected based on a trait or phenotype produced by the edit or mutation and present in the developed or regenerated plant, or a progeny plant, plant part or seed thereof. In other embodiments, the at least one edited product cell, or a progeny cell thereof, or the plant developed or regenerated from the at least one edited product cell, or a progeny cell thereof, are screened or selected based on a molecular assay.

In another aspect, the present invention provides a method for transfer of a biomolecule into a cell comprising: a) mixing a recipient plant cell culture comprising at least one recipient cell with a medium comprising at least one biomolecule to obtain a mixed cell culture comprising the recipient cell; b) wounding the recipient cell of the mixed cell culture to produce at least one product cell into which transfer of the biomolecule has occurred following said mixing and/or wounding; and c) regenerating a plant from the mixed cell culture and/or the at least one product cell, or at least one progeny cell thereof.

The present invention provides a method for transfer of a biomolecule into a cell comprising: a) wounding a recipient cell of a recipient plant cell culture; and b) mixing the recipient cell culture with a medium comprising at least one biomolecule to obtain a mixed cell culture comprising the recipient cell and to produce at least one product cell into which transfer of the biomolecule has occurred following said wounding and/or mixing. In some embodiments, the recipient plant cell culture, medium or mixed cell culture further comprises an osmoticum. In further embodiments, the osmoticum comprises polyethylene glycol (PEG). In yet further embodiments, the osmoticum comprises a sugar or sugar alcohol. In some embodiments, the recipient plant cell culture is a callus culture or cell suspension culture. In other embodiments, one or more recipient cells of the recipient plant cell culture comprise a genotype, genetic background, transgene, native allele, edit or mutation of interest. In further embodiments, the at least one product cell, or a progeny cell thereof, comprises the genotype, genetic background, transgene, native allele, edit or mutation of interest from the recipient plant cells. In some embodiments, the at least one biomolecule comprises a site-specific nuclease, a guide RNA, or one or more recombinant DNA molecules comprising a sequence encoding a site-specific nuclease and/or a sequence encoding a guide RNA. In further embodiments, the site-specific nuclease is a zinc-finger nuclease (ZFN), a meganuclease, an RNA-guided endonuclease, a TALE-endonuclease (TALEN), a recombinase, or a transposase. In some embodiments, cells of the recipient plant cell culture are dicot plant cells. In other embodiments, cells of the first and/or second plant cell cultures are monocot plant cells. The present invention further provides a product cell produced by the method described herein. In some embodiments, the product cell is a dicot plant cell. In further embodiments, the product cell is selected from the group consisting of: a tobacco, a tomato, a soybean, a canola, and a cotton plant cell. In other embodiments, the product cell is a monocot plant cell. In further embodiments, the product cell is selected from the group consisting of: a corn, a rice, a wheat, and a sorghum plant cell. A plant regenerated from the product cell produced by the method provided herein, or a progeny cell thereof is also provided. In some embodiments, the regenerated plant is a dicot plant. In further embodiments, the dicot plant is selected from the group consisting of: a tobacco, a tomato, a soybean, a canola, and a cotton plant. In other embodiments, the regenerated plant is a monocot plant. In further embodiments, the monocot plant is selected from the group consisting of: a corn, a rice, a wheat, a barley, and a sorghum plant. Seed, progeny plants, or progeny seed of the regenerated monocot and dicot plants are also provided herein. Also provided herein is a wounded mixed cell culture produced by the method described herein.

In another aspect, the present invention provides a method for transfer of a biomolecule into a cell comprising: a) wounding a recipient cell of a recipient plant cell culture; and b) mixing the recipient cell culture with a medium comprising at least one biomolecule to obtain a mixed cell culture comprising the recipient cell and to produce at least one product cell into which transfer of the biomolecule has occurred following said wounding and/or mixing, wherein the recipient plant cell culture comprises cells having a plastid genome-encoded marker gene and/or a nuclear genome-encoded marker gene. In some embodiments, the method further comprises the step of: c) screening or selecting for the at least one product cell of the mixed cell culture, or at least one progeny cell thereof, or a plant developed or regenerated from the at least one product cell, or a progeny cell thereof, based on the presence of the nuclear genome encoded marker gene and/or the plastid genome-encoded marker gene, during and/or after step (b).

In yet another aspect, the present invention provides a method for transfer of a biomolecule into a cell comprising: a) wounding a recipient cell of a recipient plant cell culture; b) mixing the recipient cell culture with a medium comprising at least one biomolecule to obtain a mixed cell culture comprising the recipient cell and to produce at least one product cell into which transfer of the biomolecule has occurred following said wounding and/or mixing; and c) screening or selecting for the at least one product cell, or a progeny cell thereof, or a plant developed or regenerated from the at least one product cell, or a progeny cell thereof, based on a selectable or screenable marker. In some embodiments, the method further comprises the step of d) regenerating a plant from the mixed cell culture and/or the at least one product cell, or at least one progeny cell thereof.

In another aspect, the present invention provides a method for transfer of a biomolecule into a cell comprising: a) wounding a recipient cell of a recipient plant cell culture; b) mixing the recipient cell culture with a medium comprising at least one biomolecule to obtain a mixed cell culture comprising the recipient cell and to produce at least one product cell into which transfer of the biomolecule has occurred following said wounding and/or mixing; and c) adding an osmoticum to the recipient plant cell culture, medium or mixed cell culture prior to, during or after step a) or step b). In some embodiments, the osmoticum comprises polyethylene glycol (PEG). In other embodiments, the osmoticum comprises a sugar or sugar alcohol.

In yet another aspect, the present invention provides a method for transfer of a biomolecule into a cell comprising: a) wounding a recipient cell of a recipient plant cell culture; b) mixing the recipient cell culture with a medium comprising at least one biomolecule to obtain a mixed cell culture comprising the recipient cell and to produce at least one product cell into which transfer of the biomolecule has occurred following said wounding and/or mixing; and c) screening or selecting for at least one edited or mutated product cell, or a progeny cell thereof, or a plant developed or regenerated from the at least one edited product cell, or a progeny cell thereof, having the edit or mutation. In some embodiments, the plant developed or regenerated from the at least one edited or mutated product cell, or a progeny cell thereof, is screened or selected based on a trait or phenotype produced by the edit or mutation and present in the developed or regenerated plant, or a progeny plant, plant part or seed thereof. In other embodiments, the at least one edited product cell, or a progeny cell thereof, or the plant developed or regenerated from the at least one edited product cell, or a progeny cell thereof, are screened or selected based on a molecular assay.

The present invention provides a method for editing a plant cell comprising: a) mixing a recipient plant cell culture comprising a recipient cell with a medium comprising at least one biomolecule to obtain a mixed cell culture comprising the recipient cell, wherein the biomolecule comprises a site-specific nuclease or a recombinant DNA molecule comprising a sequence encoding a site-specific nuclease operably linked to a first promoter; and b) wounding the recipient cell of the mixed cell culture to produce at least one edited product cell having an edit or mutation introduced in its genome by the site-specific nuclease. In some embodiments, the plant developed or regenerated from the at least one edited product cell, or a progeny cell thereof, is screened or selected based on a trait or phenotype produced by the edit or mutation and present in the developed or regenerated plant, or a progeny plant, plant part or seed thereof. In other embodiments, the at least one edited product cell, or a progeny cell thereof, or the plant developed or regenerated from the at least one edited product cell, or a progeny cell thereof, are screened or selected based on a molecular assay. In some embodiments, the recipient plant cell culture, medium or mixed cell culture further comprises an osmoticum. In some embodiments, the method further comprises the step of: c) adding an osmoticum to the recipient plant cell culture, medium or mixed cell culture prior to, during or after step a) or step b). In further embodiments, the osmoticum comprises polyethylene glycol (PEG). In yet further embodiments, the osmoticum comprises a sugar or sugar alcohol. In other embodiments, the recipient plant cell culture is a callus culture or cell suspension culture. In some embodiments, cells of the recipient plant cell culture are dicot plant cells. In further embodiments, the dicot plant cells are selected from the group consisting of tobacco, tomato, soybean, canola, and cotton cells. In other embodiments, cells of the recipient plant cell culture are monocot plant cells. In further embodiments, the monocot plant cells are selected from the group consisting of corn, rice, wheat, barley, and sorghum cells. The present invention further provides a product cell produced by the method described herein. In some embodiments, the product cell is a dicot plant cell. In further embodiments, the product cell is selected from the group consisting of: a tobacco, a tomato, a soybean, a canola, and a cotton plant cell. In other embodiments, the product cell is a monocot plant cell. In further embodiments, the product cell is selected from the group consisting of: a corn, a rice, a wheat, and a sorghum plant cell. In some embodiments, the first promoter operably linked to the sequence encoding a site-specific nuclease is a constitutive promoter, a tissue-specific or tissue-preferred promoter, a developmental stage promoter, or an inducible promoter. In other embodiments, the site-specific nuclease is a zinc-finger nuclease (ZFN), a meganuclease, an RNA-guided endonuclease, a TALE-endonuclease (TALEN), a recombinase, or a transposase. In further embodiments, the site-specific nuclease is an RNA-guided nuclease. In some embodiments, the medium further comprises a first recombinant DNA construct comprising a first transcribable DNA sequence encoding a guide RNA molecule operably linked to a promoter. In further embodiments, the promoter operably linked to the first transcribable DNA sequence is a constitutive promoter, a tissue-specific or tissue-preferred promoter, a developmental stage promoter, or an inducible promoter. In some embodiments, the medium further comprises a donor template molecule or a second recombinant DNA construct comprising a second transcribable DNA sequence encoding a donor template molecule operably linked to a promoter. In further embodiments, the donor template molecule comprises a transgene comprising a coding sequence or transcribable DNA sequence operably linked to a plant-expressible promoter. In further yet embodiments, the promoter operably linked to the second transcribable DNA sequence is a constitutive promoter, a tissue-specific or tissue-preferred promoter, a developmental stage promoter, or an inducible promoter. In some embodiments, one or more cells of the second plant cell culture comprise a recombinant DNA construct comprising a first transcribable DNA sequence encoding a guide RNA molecule operably linked to a promoter. In other embodiments, the recipient cell of the recipient plant cell culture comprises a donor template molecule or a recombinant DNA construct comprising a second transcribable DNA sequence encoding a donor template molecule operably linked to a promoter. In further embodiments, the donor template molecule comprises a transgene comprising a coding sequence or transcribable DNA sequence operably linked to a plant-expressible promoter. Also provided herein is an edited product cell produced by the method described herein. In some embodiments, the edited product cell is a is a dicot plant cell. In other embodiments, the edited product cell is a is a monocot plant cell. A plant regenerated or developed from the edited product cell produced by the method described herein is also provided by the present invention. In some embodiments, the regenerated plant is a dicot or monocot plant. Seed, progeny plants, or progeny seed of the regenerated plants are also provided herein. Also provided herein is a wounded mixed cell culture produced by the method described herein.

In another aspect, the present invention provides a method for editing a plant cell comprising: a) mixing a recipient plant cell culture comprising a recipient cell with a medium comprising at least one biomolecule to obtain a mixed cell culture comprising the recipient cell, wherein the biomolecule comprises a site-specific nuclease or a recombinant DNA molecule comprising a sequence encoding a site-specific nuclease operably linked to a first promoter; b) wounding the recipient cell of the mixed cell culture to produce at least one edited product cell having an edit or mutation introduced in its genome by the site-specific nuclease; and c) screening or selecting for the at least one edited product cell, or a progeny cell thereof, or a plant developed or regenerated from the at least one edited product cell, or a progeny cell thereof, having the edit or mutation. In some embodiments, the method further comprises the step of: d) regenerating a plant from the mixed cell culture and/or the at least one edited product cell, or at least one progeny cell thereof.

In yet another aspect, the present invention provides a method for editing a plant cell comprising: a) mixing a recipient plant cell culture comprising a recipient cell with a medium comprising at least one biomolecule to obtain a mixed cell culture comprising the recipient cell, wherein the biomolecule comprises a site-specific nuclease or a recombinant DNA molecule comprising a sequence encoding a site-specific nuclease operably linked to a first promoter; b) wounding the recipient cell of the mixed cell culture to produce at least one edited product cell having an edit or mutation introduced in its genome by the site-specific nuclease; and c) regenerating a plant from the mixed cell culture and/or the at least one edited product cell, or at least one progeny cell thereof.

The present invention provides a method for editing a plant cell comprising: a) wounding a recipient cell of a recipient plant cell culture; and b) mixing the recipient plant cell culture with a medium comprising at least one biomolecule to obtain a mixed cell culture comprising the recipient cell, wherein the biomolecule comprises a site-specific nuclease or a recombinant DNA molecule comprising a sequence encoding a site-specific nuclease operably linked to a first promoter, to produce at least one edited product cell having an edit or mutation introduced in its genome by the site-specific nuclease. In some embodiments, the plant developed or regenerated from the at least one edited product cell, or a progeny cell thereof, is screened or selected based on a trait or phenotype produced by the edit or mutation and present in the developed or regenerated plant, or a progeny plant, plant part or seed thereof. In other embodiments, the at least one edited product cell, or a progeny cell thereof, or the plant developed or regenerated from the at least one edited product cell, or a progeny cell thereof, are screened or selected based on a molecular assay. In some embodiments, the recipient plant cell culture, medium or mixed cell culture further comprises an osmoticum. In some embodiments, the method further comprises the step of: c) adding an osmoticum to the recipient plant cell culture, medium or mixed cell culture prior to, during or after step a) or step b). In further embodiments, the osmoticum comprises polyethylene glycol (PEG). In other embodiments, the recipient plant cell culture is a callus culture or cell suspension culture. In some embodiments, cells of the recipient plant cell culture are dicot plant cells. In further embodiments, the dicot plant cells are selected from the group consisting of tobacco, tomato, soybean, canola, and cotton cells. In other embodiments, cells of the recipient plant cell culture are monocot plant cells. In further embodiments, the monocot plant cells are selected from the group consisting of corn, rice, wheat, barley, and sorghum cells. The present invention further provides a product cell produced by the method described herein. In some embodiments, the product cell is a dicot plant cell. In further embodiments, the product cell is selected from the group consisting of: a tobacco, a tomato, a soybean, a canola, and a cotton plant cell. In other embodiments, the product cell is a monocot plant cell. In further embodiments, the product cell is selected from the group consisting of: a corn, a rice, a wheat, and a sorghum plant cell. In some embodiments, the first promoter operably linked to the sequence encoding a site-specific nuclease is a constitutive promoter, a tissue-specific or tissue-preferred promoter, a developmental stage promoter, or an inducible promoter. In other embodiments, the site-specific nuclease is a zinc-finger nuclease (ZFN), a meganuclease, an RNA-guided endonuclease, a TALE-endonuclease (TALEN), a recombinase, or a transposase. In further embodiments, the site-specific nuclease is an RNA-guided nuclease. In some embodiments, the medium further comprises a first recombinant DNA construct comprising a first transcribable DNA sequence encoding a guide RNA molecule operably linked to a promoter. In further embodiments, the promoter operably linked to the first transcribable DNA sequence is a constitutive promoter, a tissue-specific or tissue-preferred promoter, a developmental stage promoter, or an inducible promoter. In some embodiments, the medium further comprises a donor template molecule or a second recombinant DNA construct comprising a second transcribable DNA sequence encoding a donor template molecule operably linked to a promoter. In further embodiments, the donor template molecule comprises a transgene comprising a coding sequence or transcribable DNA sequence operably linked to a plant-expressible promoter. In further yet embodiments, the promoter operably linked to the second transcribable DNA sequence is a constitutive promoter, a tissue-specific or tissue-preferred promoter, a developmental stage promoter, or an inducible promoter. In some embodiments, one or more cells of the second plant cell culture comprise a recombinant DNA construct comprising a first transcribable DNA sequence encoding a guide RNA molecule operably linked to a promoter. In other embodiments, the recipient cell of the recipient plant cell culture comprises a donor template molecule or a recombinant DNA construct comprising a second transcribable DNA sequence encoding a donor template molecule operably linked to a promoter. In further embodiments, the donor template molecule comprises a transgene comprising a coding sequence or transcribable DNA sequence operably linked to a plant-expressible promoter. Also provided herein is an edited product cell produced by the method described herein. In some embodiments, the edited product cell is a is a dicot plant cell. In other embodiments, the edited product cell is a is a monocot plant cell. A plant regenerated or developed from the edited product cell produced by the method described herein is also provided by the present invention. In some embodiments, the regenerated plant is a dicot or monocot plant. Seed, progeny plants, or progeny seed of the regenerated plants are also provided herein. Also provided herein is a wounded mixed cell culture produced by the method described herein.

In another aspect, the present invention provides a method for editing a plant cell comprising: a) wounding a recipient cell of a recipient plant cell culture; b) mixing the recipient plant cell culture with a medium comprising at least one biomolecule to obtain a mixed cell culture comprising the recipient cell, wherein the biomolecule comprises a site-specific nuclease or a recombinant DNA molecule comprising a sequence encoding a site-specific nuclease operably linked to a first promoter, to produce at least one edited product cell having an edit or mutation introduced in its genome by the site-specific nuclease; and c) screening or selecting for the at least one edited product cell, or a progeny cell thereof, or a plant developed or regenerated from the at least one edited product cell, or a progeny cell thereof, having the edit or mutation. In some embodiments, the method further comprises the step of: d) regenerating a plant from the mixed cell culture and/or the at least one edited product cell, or at least one progeny cell thereof.

In another aspect, the present invention provides a method for editing a plant cell comprising: a) wounding a recipient cell of a recipient plant cell culture; b) mixing the recipient plant cell culture with a medium comprising at least one biomolecule to obtain a mixed cell culture comprising the recipient cell, wherein the biomolecule comprises a site-specific nuclease or a recombinant DNA molecule comprising a sequence encoding a site-specific nuclease operably linked to a first promoter, to produce at least one edited product cell having an edit or mutation introduced in its genome by the site-specific nuclease; and c) regenerating a plant from the mixed cell culture and/or the at least one edited product cell, or at least one progeny cell thereof.

### BRIEF DESCRIPTION OF THE DRAWINGS

FIG. 1: Shows the components in the GFP reporter construct inserted into the nuclear genome of transgenic corn line A. In the 5′ to 3′ direction, there is an enhanced CaMV 35S promoter with an HSP70 intron in the 5′ untranslated region, a nptII selectable marker gene cassette flanked by two lox sites, followed by a green fluorescent protein (GFP) encoding gene. In the absence of Cre recombinase enzyme, GFP is not functionally expressed due to the intervening nptII gene between the 35S promoter and the GFP coding sequence. However, in the presence of Cre recombinase enzyme, the nptII gene is excised due to the flanking lox sites, which results in high levels of GFP expression.

FIG. 2: FIGS. 2A-C show images of the first GFP-positive callus pieces that were identified after 3 weeks of culture. FIGS. 2D and 2E show images of GFP expression in leaves obtained from plants regenerated from GFP-positive calli.

FIG. 3: Shows PCR results using primers designed to amplify the GFP reporter construct. Genomic DNA was isolated from leaf tissue taken from Plants 1-3, which were regenerated from GFP-positive callus, and Plant 4, which was the GFP-negative control plant, using the CTAB method known in the art. PCR reactions were carried out and the PCR products from these reactions were resolved in 1% agarose gel. Cre-excision of the nptII gene cassette was confirmed by the presence of a ˜0.97 kb band for the excised DNA fragment, as compared to a ˜2.18 kb band for unexcised DNA fragment.

FIG. 4: FIG. 4A shows an image of the regenerated GFP-positive (Plants 1-3) and negative (Plant 4) plants. FIG. 4B shows the GFP expression visualized under a blue light in a tassel spikelet of Plant 2, which is a GFP positive plant. FIG. 4C shows the GFP expression in a tassel spikelet from Plant 4, the GFP-negative control.

FIG. 5: Shows GFP expression in cells cultured for at least three days in medium 1074.

FIGS. 5A and 5B show that GFP expression was only observed in plates containing the blended callus suspension that had been treated with PEG after three days of culturing. FIG. 5C show that GFP expression was only observed in plates containing the blended callus suspension that had been treated with PEG after six days of culturing. No GFP expression was found in plates where callus with Cre was not treated with PEG.

### DETAILED DESCRIPTION

The present disclosure provides novel methods and compositions for introducing, transferring or delivering (i.e., transferring) genetic material, polynucleotides, DNA, proteins, nucleases and/or ribonucleoproteins into plant cells and tissues to create cells or plants with a desired mutation, edit, genotype and/or phenotype or trait. There is a need in the art for an efficient and effective technology for introducing, transferring or delivering (i.e., transferring) biomolecules into one or more plant cells to generate a genetic change, mutation, or edit in the one or more plant cells to produce a desired genotype, phenotype or trait in a plant developed or regenerated from the one or more plant cells. As used herein, the transfer, delivery and/or introduction of a biomolecule into a recipient cell is collectively referred to as “transfer” or “transferring” of the biomolecule into the recipient cell, and likewise a biomolecule being transferred, delivered and/or introduced into a recipient cell is collectively referred to as the biomolecule being “transferred” into the recipient cell.

The present disclosure describes methods of introducing, transferring or delivering (i.e., transferring) a biomolecule into a plant cell or a population of plant cells, such as a parental plant cell, which may be growing in vitro, for instance as a callus or cell suspension culture, which may be accompanied and aided by wounding of those cells or tissues in culture. Such an introduction, delivery or transfer (i.e., transfer) of a biomolecule may result in the creation of a mutation, edit or other genetic change that leads to a new genotype, characteristic, phenotype, and/or trait in the plant cell or population of plant cells or in a plant or plant part developed, grown or regenerated from such plant cells. Without being bound by theory, wounding of the plant cells, for instance by chopping with a razor blade, knife, or other sharp instrument, or by sonication, vortexing, shaking, blending, electroporation, or other means, may disrupt or create openings or pores in the plant cell wall and/or plasma membrane of the plant cell(s) that can allow for biomolecules present in the environment or surroundings of the plant cell(s) (e.g., media, solution, or mixture) to enter, become introduced, delivered or transferred into the plant cell(s) from the environment or surroundings to form a product cell having the biomolecule(s) within its cytoplasm, cytosol, organelle or nucleus. Without being bound by theory, the plasma membranes and/or cell wall may be disrupted or opened to a limited extent such that the plant cell remains viable and able to divide and form progeny or daughter plant cells that can continue to further divide, develop and differentiate to form a plant or plant part. According to some embodiments, the biomolecule may have a signal or targeting sequence or tag, which may be fused to the biomolecule, that functions to target the biomolecule to a particular compartment (e.g., nucleus, chloroplast, mitochondria, etc.) of the plant product cell. According to some embodiments, one or more agent(s) that promote(s) cell permeation may also be utilized, such as the use of different osmoticums (e.g., polyethylene glycol (PEG), sugars, sugar alcohols, etc.), presence of high calcium (or other cation) concentration, higher pH, and/or other compounds and conditions that are known to promote cell membrane fusion in other methods, which may help introduce, deliver or transfer (i.e., transfer) a biomolecule(s) into a plant cell. A product cell or a population of product cells produced by present methods may then be grown, developed and/or regenerated, perhaps with screening or selection for a marker gene (transgenic or non-transgenic) present in the product cell or progeny thereof, or by the creation of a new trait, genotype or marker expression, or by the molecular detection of a mutation, edit or other genetic change. Plants grown or regenerated from these product cells may then be identified, isolated or selected based on a novel trait, phenotype or combination of traits or phenotypes, such as one or more genetic traits and/or markers.

A method is provided herein of introducing, delivering or transferring (i.e., transferring) one or more biomolecules into a target or recipient plant cell, a population of target or recipient plant cells, or a mixed population of target or recipient cells from two or more parental types, varieties, germplasms or genotypes, which may be growing in vitro, such as, for example, as callus or a cell suspension Such transfer of biomolecules into a target or recipient plant cell is further promoted by wounding the target or recipient callus cells or suspension of cells or clumps or clusters of cells. Without being bound theory, the transfer of biomolecule(s) into the target or recipient plant cells according to present methods may not require protoplasting, formation of plasmodesmata, nor successful grafting of different plant cells or tissues.

As described in the examples below, non-organized growing corn or maize tissue (callus), having a GFP reporter construct disrupted by a nptII marker gene cassette flanked by lox sites were wounded and mixed with a Cre recombinase protein enzyme in the surrounding medium. Wounding the callus corn cells allowed the Cre recombinase enzyme to enter the cells, particularly in the presence of one or more osmoticum(s) or osmoticum agent(s), and cause excision of the nptII marker gene by acting on the flanking lox sites to bring the 35S promoter into proximity of the GFP coding sequence leading to detectable fluorescence by expression of the GFP reporter. GFP positive cells and tissues were produced by these methods indicating effective introduction, transfer or delivery (i.e., transfer) of the Cre recombinase into the target or recipient callus cells. In addition to GFP expression, molecular analysis further confirmed the presence of the Cre recombinase in the target or recipient callus cells by excision of the nptII gene cassette based on PCR fragment size.

This disclosure provides methods for producing a mutated, edited or genetically modified plant cell(s) or population of plant cells, and compositions of (or comprising) such mutated, edited or genetically modified plant cell(s) (or product cell(s)) or population of plant cells or product cell(s), by delivering one or more biomolecule(s) to a wounded target or recipient plant cell(s) or population of plant cells, which may be in the presence of one or more osmoticum agents. The recipient and/or product cells of these methods may comprise one or more unique or different transgenes, markers, recombination events, insertions, deletions, mutations, edits, etc. These methods can allow for effective introduction, transfer, or delivery (i.e., transfer) of one or more protein(s), ribonucleoprotein(s), polynucleotide(s), DNA molecule(s), genetic material(s), or other biomolecule(s), or any combination thereof, to make a mutation, edit or other genetic change to the recipient or target cells. In certain embodiments, the target, recipient and/or product plant cells are dicot plant cells, such as from tobacco, tomato, soybean, cotton, canola, alfalfa, sugar beets, Arabidopsis, or other fruits and vegetables. In other embodiments the target, recipient and/or product plant cells may be from monocot plants, such as from corn, wheat, rice, sorghum, barley, or other cereal plants and vegetables. The target, recipient and/or product cells may be from an in vitro grown cell culture, such as a cell suspension or a callus culture, which may be a regenerable callus culture. It is also possible that a target, recipient and/or product cell, callus or cell suspension may be non-regenerable, although it is generally preferable for the target, recipient and/or product cell(s) to be regenerable into a plant or plant part.

As used herein, a “product cell” is a cell produced by a method or experiment of the present disclosure that has one or more biomolecule(s) introduced, transferred or delivered (i.e., transferred) from its environment or surroundings, and which may have one or more mutation(s), edit(s) and/or other genetic change(s). In some embodiments, a “product cell” refers to a cell produced by a method or experiment of the present disclosure that has an edit or targeted (site-directed) insertion introduced by a site-specific nuclease delivered from its environment or surrounding media, solution, etc., or by a site-specific nuclease and/or guide RNA expressed from a polynucleotide(s) introduced into a recipient cell, or by a site-specific nuclease expressed from a polynucleotide(s) introduced into a recipient cell in conjunction with a guide RNA expressed from a construct or expression cassette already present or preexisting in the recipient cell. An “edit” refers to a change (e.g., insertion, deletion, substitution, inversion, etc.) in the nuclear genomic sequence of a resulting or product plant cell, and in a plant developed or regenerated from such a product plant cell, or a progeny plant thereof, and in a plant part or seed from any of the foregoing, relative to the corresponding genomic sequence of an otherwise identical plant cell or plant, such as a parental or recipient plant cell or plant, which was not been subjected to such “editing”. Such an edit may be within an intergenic region of a plant genome or a genic region of a plant genome, such as at or near a native gene or transgene (e.g., in an enhancer, promoter, splice site, coding sequence, exon, intron, 5′ or 3′ untranslated region (UTR), terminator, etc.) present in the recipient cell, to affect the expression and/or activity of such gene or transgene. A product cell and plant, and progeny thereof, may have a genotype, traits and/or phenotypes, including morphological and reproductive traits, that are similar or identical to the recipient plant or plant cell due to a relatively minor genetic modification produced by the biomolecule being introduced, delivered, or transferred (i.e., transferred), into the recipient cell, and the product cell retaining most or all of the nuclear, mitochondrial and/or plastid genomes, cellular components and genetic background of the recipient cell, with the exception of the mutation(s), edit(s), and/or genetic modification(s) produced by transfer of the one or more biomolecule(s) into the recipient cell(s).

Wounding may be accomplished by methods known in the art. For instance, chopping or cutting of cells with a razor blade, knife or other sharp instrument, and wounding by sonication, can be effective. Wounding may also be achieved by vortexing, shaking, blending, electroporation, or other mechanical means. Without being bound by theory, wounding of plant cells may create holes or pores in plant cell walls, increase the permeability of the plasma membrane, and/or increase the uptake of the surrounding medium by the recipient cell(s). In the process of wounding or repair, a plant cell may take up and keep some of the contents of surrounding medium, mixture, or solution, including any biomolecule(s) or other components present in the surrounding medium or environment.

Once a wounded cell culture has been produced and exposed or mixed with a medium, solution or mixture containing one or more biomolecule(s), selection or screening for the presence of a desired genetic modification, trait or marker, or a desired combination of genetic traits and/or markers, may be performed, during and/or after growth and regeneration of the product cell culture, and progeny thereof, and/or plants or plant parts regenerated from the foregoing, to select or screen for cells, plants or plant parts having the biomolecule and/or at least one desired mutation, edit and/or genetic modification. In certain embodiments, selection is imposed after wounding and/or exposure of the recipient cell(s) to the biomolecule(s), which may occur immediately after wounding the recipient cell culture and/or later (e.g., even while the wounded population of cells is being prepared). Selection may occur, for example, by incorporation of an effective amount of a selective agent within one or more culture media.

As used herein, a “biomolecule” refers to any biological molecule that may be introduced into a wounded recipient plant cell, perhaps along with one or more other biomolecule(s), according to the methods described herein. A biomolecule (or a combination of two or more biomolecules) will generally be a biological molecule (or a combination of two or more biological molecules) that can directly or indirectly cause or create one or more mutation(s), edit(s), and/or other genetic change(s) to the genome of a recipient plant cell when introduced, delivered or transferred (i.e., transferred) into the recipient plant cell. Examples of biomolecules can include a nuclease, such as a site-specific nuclease, a recombinase, a ribonucleoprotein, a guide RNA, or a recombinant polynucleotide or DNA molecule comprising a sequence(s) encoding any one or more of the foregoing. A biomolecule can be a site-specific nuclease is a zinc-finger nuclease (ZFN), a meganuclease, an RNA-guided endonuclease, a TALE-endonuclease (TALEN), a recombinase, or a transposase, a guide RNA, or a donor DNA template molecule, or a recombinant polynucleotide or DNA molecule comprising a sequence(s) encoding any one or more of the foregoing. For clarity, one or more biomolecules, two or more biomolecules, etc., may be transferred to a recipient cell according to present methods, and/or a recipient cell may already have or express one or more biomolecules prior to transfer of one or more biomolecules into the recipient cell.

In certain embodiments, it may be desirable to utilize transgenic or mutant traits or markers for selection or screening. Such traits may, for instance, include antibiotic or herbicide tolerance, such as resistance to kanamycin, streptomycin, spectinomycin, hygromycin, glyphosate, glufosinate, dicamba, etc. These traits may be plastid-encoded or nuclear-encoded. Other traits useful for selection or screening may include those which result in production of a visually detectable phenotype or product, such as GUS, GFP, or a carotenoid, such as phytoene, etc. Such traits or markers may be introduced by a biomolecule that is a polynucleotide comprising a selectable marker gene or expression cassette, or may be present in the recipient cell(s) prior to introduction of the one or more biomolecule(s).

Wounding a recipient or target plant cell or a population of recipient or target cells growing in vitro, before or after mixing the recipient or target plant cell(s) with a biomolecule and possibly before or after mixing the recipient or target plant cell(s) with an osmoticum, can result in a product cell(s) comprising the biomolecule inside the product cell(s), which can produce one or more mutation(s), edit(s) and/or other genetic change(s) in the genome(s) of the product cell(s).

The term “transgene” refers to an exogenously introduced DNA molecule or construct into at least one cell of an organism that is incorporated into an organism's genome as a result of human intervention, such as by plant transformation methods. As used herein, the term “transgenic” refers to a material comprising a transgene or recombinant expression cassette or construct. For example, a “transgenic plant” refers to a plant comprising a transgene or recombinant expression cassette or construct in its genome, and a “transgenic trait” refers to a characteristic or phenotype of a plant caused, conveyed or conferred by the presence of a transgene or recombinant expression cassette or construct incorporated into the plant genome. As a result of such genomic alteration, the transgenic plant is something distinctly different from a related wild-type plant. According to many embodiments, a transgene may comprise a coding sequence or transcribable DNA sequence operably linked to a promoter, such as a plant-expressible promoter. A plant-expressible promoter may express in one or more plant cells, such as a recipient and/or product cell according to the present disclosure. A plant-expressible promoter may be a constitutive promoter, a tissue-specific or tissue-preferred promoter, a developmental stage promoter, or an inducible promoter. According to many embodiments, the transcribable DNA sequence or coding sequence of the transgene may encode a RNA or protein of interest, such as a structural protein, enzyme, RNA suppression element or guide RNA for a site-specific nuclease. According to some embodiments, a coding sequence of a transgene may comprise a coding sequence of a marker gene, which may be present in the nuclear or plastid genome. The marker gene may be a selectable marker gene or a screenable marker gene as further described herein. According to some embodiments, a coding sequence of a transgene may encode a site-specific nuclease.

As used herein and according to its commonly understood meaning, a “control” means an experimental control designed for comparison purposes, which is typically similar to an experimental or test subject except for the one or more differences or modifications being tested or studied. For example, a control plant may be a plant of the same or similar type as the experimental or test plant having one or more modifications of interest (e.g., a transgene, mutation, edit, etc.) that does not contain the modification(s) present in the experimental plant.

### Transgenic, Mutated or Edited Plants

An aspect of the invention includes transgenic plant cells, transgenic plant tissues, transgenic plants, and transgenic seeds that comprise a transgene or recombinant DNA molecule, wherein the transgene may be present in a recipient cell before wounding and/or introduction of a biomolecule or introduced by the biomolecule according to the present methods. These cells, tissues, plants, and seeds comprising the recombinant DNA molecules, transgenes, constructs, cassettes, etc., may exhibit tolerance to a selection agent, such as one or more herbicides or antibiotics, or provide a screenable marker or another phenotype or trait of interest, such as an agronomic trait of interest. According to some embodiments, a plant cell used or generated in the methods or experiments of the present disclosure may be a transgenic plant cell, which may be derived from a transgenic plant.

Any suitable transformation methods may be used to produce a transgenic cell, plant part or plant, and a transgenic recipient cell may be derived from such transgenic cell, plant part or plant. Recipient cell(s) used in methods of the present disclosure may include transgenic plant cell(s) produced by these methods. Methods for transformation of plant cells include any method by which DNA can be introduced into a cell (for example, where a recombinant DNA construct is stably integrated into a plant chromosome). Methods of plant transformation are known in the art. Methods for introducing a recombinant DNA construct into plants may include bacterially-mediated (or Agrobacterium-mediated) transformation or particle-bombardment techniques for transformation, both of which are well known to those of skill in the art. Another method that may be used for introducing a recombinant DNA construct as a transgene into plants is insertion of a recombinant DNA construct into a plant genome at a pre-determined site by methods of site-directed integration. Site-directed integration may be accomplished by any method known in the art, for example, by use of zinc-finger nucleases, engineered or native meganucleases, TALE-endonucleases, or an RNA-guided endonuclease (for example, a CRISPR/Cas9 system) in combination with a template DNA for making the genomic insertion at a desired target site. Thus site-directed integration may be used to introduce a transgene at a desired location in the genome. Methods for culturing explants and plant parts, as well as methods for selecting and regenerating plants in culture, are also known in the art. Alternatively, methods of the present disclosure may be used to deliver or insert a transgene into the genome of a recipient plant cell by introduction, transfer or delivery (i.e., transfer) of one or more biomolecule(s) into the recipient plant cell, which function to create the desired mutation, edit, transgene insertion or other genetic modification.

Once a plant cell is transformed by either a known technique, such as bacterial or Agrobacterium-meditated transformation, particle bombardment, or genome editing including site-directed integration or using a biomolecule delivery method of the present disclosure, transgenic plants can be developed or regenerated from a transformed plant cell, tissue or plant part by any known culturing methods for plant cells, tissues or explants. A transgenic plant homozygous with respect to a mutation, edit, allele or transgene (that is, having two copies of the mutation, edit, allele or transgene) can be obtained by self-pollinating (selfing) a mutated, edited or transgenic plant that contains a single mutation, edit or transgene allele with itself, for example an R0 plant, to produce R1 seed. Transgenic, mutated or edited offspring, such as plants grown from R1 seed, can be tested for zygosity using any known zygosity assay, such as by using a SNP assay, DNA sequencing, thermal amplification or PCR, and/or Southern blotting that allows for the distinction between heterozygotes, homozygotes and wild type, or by observing or selecting for a phenotype or trait expected based on the zygosity.

Plants and progeny that contain a novel mutation(s), edit(s), transgene(s) and/or trait(s), or a novel combination thereof, as provided herein may be used with any breeding methods that are commonly known in the art. Methods for breeding or crossing plants that are commonly used for different traits and crops are known to those of skill in the art. Methods of the present disclosure may be used as an additional breeding tool by introducing a biomolecule(s) into a recipient plant cell(s) having a desirable genetic background, germplasm or genotype, except for an additional mutation, edit, transgene, or other genetic modification to be caused or created by the introduction of the biomolecule(s) into the recipient plant cell(s). Indeed, methods of the present disclosure may be used to introduce mutation, edit, transgene, or other genetic modification at a site in the genome that is closely linked or associated with another desirable trait, marker, gene or sequence in the genome, which may be difficult to combine through normal breeding, introgression and backcross conversion. A plant genotype into which a transgenic trait has been introgressed may be referred to as a backcross converted genotype, line, inbred, or hybrid. Similarly, a plant genotype lacking the desired transgenic trait, etc., may be referred to as an unconverted genotype, line, inbred, or hybrid.

Aspects of the present disclosure may be used in breeding or introgression efforts as a replacement for crossing plants through sexual reproduction to allow for a combination of genetic traits and/or cellular components in combined product cells, which may be developed or regenerated into plants having a desired combination or introduction of traits. Such plants may be identified or selected based on the presence of one or more mutations, edits, transgenes, markers, traits or phenotypes. To confirm the presence of the transgene(s), mutation(s), edit(s) or other genetic change(s) or trait(s) in a plant, plant part or seed or progeny thereof, such as a plant regenerated from a product cell as provided herein, or a plant part, seed or progeny thereof, a variety of assays may be performed and used. Such assays can include, for example, molecular biology assays, such as Southern and northern blotting, PCR, and DNA sequencing; biochemical assays, such as detecting the presence of a protein product, for example, by immunological means (ELISAs and western blots) or by enzymatic function; plant part assays, such as leaf or root assays; and also by analyzing a phenotype or trait of the whole plant.

### Gene Editing and Recombination

The ability to introduce, transfer or deliver (i.e., transfer) a biomolecule(s) into a recipient cell or recipient cell culture or population of recipient cells according to present methods provides the potential to introduce, transfer or deliver (i.e., transfer) RNA, protein and/or other molecules or factors present in the surrounding medium into the recipient plant cell. These biomolecules may be introduced, transferred or delivered into a recipient cell without becoming integrated into the genomic DNA of the recipient cell. Thus, RNA and/or protein may be introduced, transferred or delivered into a recipient cell according to present methods and exert an activity, effect or change on the recipient cell. The introduced, transferred or delivered RNA, protein or other biomolecule, or a combination thereof, may be present in the recipient cell only transiently, although while present in the recipient cell the biomolecule(s) may cause one or more genetic change(s) to the genome of the recipient cell. Thus, the RNA, protein and/or other biomolecule(s) may only be present in the recipient cell for a limited time depending on its starting concentration in the recipient cell following its transfer into the recipient cell from the surrounding medium and its stability or half-life in the recipient cell.

The ability to deliver RNA and/or protein to a recipient cell, without transforming, integrating or incorporating a transgene(s) encoding the RNA and/or protein into the recipient cell genome, makes it possible to make changes to a non-transgenic recipient cell genome (i.e., without transforming the genome of the recipient cell with transgene) by delivering the RNA and/or protein to the target or recipient cell from its surrounding medium. In addition to Cre recombinase, other enzymes can be delivered to a recipient cell to make changes to the recipient cell genome or DNA. According to some embodiments, a site-specific nuclease, such as a zinc-finger nuclease, a meganuclease, an RNA-guided nuclease, a TALE-nuclease, a recombinase, a transposase, or any combination thereof, may be introduced, transferred or delivered (i.e., transferred) into a recipient cell via a method of the present disclosure, which may involve wounding the recipient cells and/or exposing them to an osmoticum. In some embodiments, the RNA-guided nuclease is a CRISPR associated nuclease (non-limiting examples of CRISPR associated nucleases include, for example, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, homologs thereof, or modified versions thereof). In some embodiments, an RNA-guided nuclease and a guide RNA, or one or more DNA molecule(s) encoding one or both of the RNA-guided nuclease and guide RNA, are delivered to a recipient cell to make changes to the recipient cell DNA. In some embodiments, a RNA-guided nuclease, or a DNA molecule encoding a RNA-guided nuclease, is delivered to a recipient cell already expressing a guide RNA, which complexes with the RNA-guided nuclease to make changes to the recipient cell DNA. In some embodiments, a guide RNA, or a DNA molecule encoding a guide RNA, is delivered to a recipient cell already expressing the RNA-guided nuclease which complexes with the guide RNA to make changes to the recipient cell DNA. In some embodiments, the recipient cell may further comprise a donor DNA sequence. In some embodiments, the donor DNA sequence is a template for templated editing. In other embodiments, the donor DNA sequence comprises a transgene or recombinant DNA construct. A mutated, edited or transgenic product cell is generated by introduction, transfer or delivery (i.e., transfer) of a site-specific nuclease into a recipient cell, which may be regenerated into a plant having the mutation, edit or transgene in its genome, and progeny plants, plant parts and seeds can also be derived from the regenerated plant. In many embodiments, plants regenerated from the mutated, edited or transgenic product cell may be genetically and phenotypically similar to the plants from which the recipient cell was derived, except for any trait(s) and/or phenotype(s) that are caused by the genomic edit or mutation or transgene.

According to many of these embodiments, a method is provided for mutating or editing a plant cell comprising: mixing or combining a recipient plant cell culture with at least one biomolecule, wherein the at least one biomolecule may be present in a medium surrounding the recipient cells, wherein one or more cells of the recipient plant cell culture comprise a recombinant DNA transgene comprising a sequence encoding a site-specific nuclease operably linked to a first promoter; and wounding the cells of the recipient cell culture to produce at least one edited product cell having an edit or mutation introduced in its genome by the site-specific nuclease. Such methods may also comprise screening or selecting for the at least one edited product cell, or a progeny cell thereof, or a plant developed or regenerated from the at least one edited product cell, or a progeny cell thereof, having the edit or mutation, which may be based on a molecular assay or a trait or phenotype produced by the edit or mutation and present in a plant developed or regenerated from the edited product cell or a progeny cell thereof, or present in a progeny plant, plant part or seed thereof. In these methods, the recipient cell cultures can be callus cultures or cell suspension cultures. These methods may further comprise regenerating a plant from the at least one edited product cell, or at least one progeny cell thereof. The plant cells used in these methods may be monocot or dicot plant cells.

According to some embodiments, the biomolecule may be a polynucleotide comprising a first recombinant DNA construct comprising a first transcribable DNA sequence encoding a guide RNA molecule operably linked to a promoter. According to some embodiments, the biomolecule may be a polynucleotide comprising a second recombinant DNA construct comprising a second transcribable DNA sequence encoding a donor template molecule operably linked to a promoter. According to some embodiments, one or more of the recipient plant cells in these methods may further comprise a first recombinant DNA construct comprising a first transcribable DNA sequence encoding a guide RNA molecule operably linked to a promoter. According to some embodiments, one or more of the recipient plant cells in these methods may further comprise a second recombinant DNA construct comprising a second transcribable DNA sequence encoding a donor template molecule operably linked to a promoter.

Further provided are transgenic, mutated or edited plant cells produced by these methods, and progeny cells thereof, which may be monocot or dicot plant cells, and which may each be further developed or regenerated into a transgenic, mutated or edited plant. A seed or plant part of a developed or regenerated plant, or a progeny plant thereof, is also provided. In addition, the wounded recipient cells used in present methods and product cells produced by these methods, are further provided.

A site-specific nuclease provided herein may be selected from the group consisting of a zinc-finger nuclease (ZFN), a meganuclease, an RNA-guided endonuclease, a TALE-endonuclease (TALEN), a recombinase, a transposase, or any combination thereof. See, e.g., Khandagale, K. et al., “Genome editing for targeted improvement in plants,” Plant Biotechnol Rep 10: 327-343 (2016); and Gaj, T. et al., “ZFN, TALEN and CRISPR/Cas-based methods for genome engineering,” Trends Biotechnol. 31(7): 397-405 (2013), the contents and disclosures of which are incorporated herein by reference. A recombinase may be a serine recombinase attached to a DNA recognition motif, a tyrosine recombinase attached to a DNA recognition motif or other recombinase enzyme known in the art. A recombinase or transposase may be a DNA transposase or recombinase attached to a DNA binding domain. A tyrosine recombinase attached to a DNA recognition motif may be selected from the group consisting of a Cre recombinase, a Flp recombinase, and a Tnp 1 recombinase. According to some embodiments, a Cre recombinase or a Gin recombinase may be tethered to a zinc-finger DNA binding domain. In another embodiment, a serine recombinase attached to a DNA recognition motif provided herein is selected from the group consisting of a PhiC31 integrase, an R4 integrase, and a TP-901 integrase. In another embodiment, a DNA transposase attached to a DNA binding domain provided herein is selected from the group consisting of a TALE-piggyBac and TALE-Mutator.

According to embodiments of the present disclosure, an RNA-guided endonuclease may be selected from the group consisting of a Cas9 or a Cpf1. According to other embodiments of the present disclosure, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, and homologs or modified versions thereof, Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo) and homologs or modified versions thereof. According to some embodiments, an RNA-guided endonuclease may be a Cas9 or Cpf1 enzyme. For RNA-guided endonucleases, a guide RNA (gRNA) molecule may be further provided to direct the endonuclease to a target site in the genome of the plant via base-pairing or hybridization to cause a DSB or nick at or near the target site. The gRNA may be transformed or introduced into a recipient plant cell or tissue as a gRNA molecule, or as a recombinant DNA molecule, construct or vector comprising a transcribable DNA sequence encoding the guide RNA operably linked to a promoter or plant-expressible promoter. The promoter may be a constitutive promoter, a tissue-specific or tissue-preferred promoter, a developmental stage promoter, or an inducible promoter. As understood in the art, a “guide RNA” may comprise, for example, a CRISPR RNA (crRNA), a single-chain guide RNA (sgRNA), or any other RNA molecule that may guide or direct an endonuclease to a specific target site in the genome. A “single-chain guide RNA” (or “sgRNA”) is a RNA molecule comprising a crRNA covalently linked a tracrRNA by a linker sequence, which may be expressed as a single RNA transcript or molecule. The guide RNA comprises a guide or targeting sequence that is identical or complementary to a target site within the plant genome, such as at or near a gene. A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu, X. et al., “Target specificity of the CRISPR-Cas9 system,” Quant Biol. 2(2): 59-70 (2014), the content and disclosure of which is incorporated herein by reference. The genomic PAM sequence on the sense (+) strand adjacent to the target site (relative to the targeting sequence of the guide RNA) may comprise 5′-NGG-3′. However, the corresponding sequence of the guide RNA (i.e., immediately downstream (3′) to the targeting sequence of the guide RNA) may generally not be complementary to the genomic PAM sequence. The guide RNA may typically be a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

In addition to the guide sequence, a guide RNA may further comprise one or more other structural or scaffold sequence(s), which may bind or interact with an RNA-guided endonuclease. Such scaffold or structural sequences may further interact with other RNA molecules (e.g., tracrRNA). Methods and techniques for designing targeting constructs and guide RNAs for genome editing and site-directed integration at a target site within the genome of a plant using an RNA-guided endonuclease are known in the art.

Several site-specific nucleases, such as recombinases, zinc finger nucleases (ZFNs), meganucleases, and TALENs, are not RNA-guided and instead rely on their protein structure to determine their target site for causing the DSB or nick, or they are fused, tethered or attached to a DNA-binding protein domain or motif. The protein structure of the site-specific nuclease (or the fused/attached/tethered DNA binding domain) may target the site-specific nuclease to the target site. According to many of these embodiments, non-RNA-guided site-specific nucleases, such as recombinases, zinc finger nucleases (ZFNs), meganucleases, and TALENs, may be designed, engineered and constructed according to known methods to target and bind to a target site at or near the genomic locus of an endogenous gene of a plant to create a DSB or nick at such genomic locus to knockout or knockdown expression of the gene via repair of the DSB or nick, which may lead to the creation of a mutation or insertion of a sequence at the site of the DSB or nick, through cellular repair mechanisms, which may be guided by a donor template molecule.

In an aspect, a targeted genome editing technique described herein may comprise the use of a recombinase. In some embodiments, a tyrosine recombinase attached, etc., to a DNA recognition domain or motif may be selected from the group consisting of a Cre recombinase, a Flp recombinase, and a Tnp 1 recombinase. In an aspect, a Cre recombinase or a Gin recombinase provided herein may be tethered to a zinc-finger DNA binding domain. The Flp-FRT site-directed recombination system may come from the 2μ plasmid from the baker's yeast Saccharomyces cerevisiae. In this system, Flp recombinase (flippase) may recombine sequences between flippase recognition target (FRT) sites. FRT sites comprise 34 nucleotides. Flp may bind to the “arms” of the FRT sites (one arm is in reverse orientation) and cleaves the FRT site at either end of an intervening nucleic acid sequence. After cleavage, Flp may recombine nucleic acid sequences between two FRT sites. Cre-lox is a site-directed recombination system derived from the bacteriophage P1 that is similar to the Flp-FRT recombination system. Cre-lox can be used to invert a nucleic acid sequence, delete a nucleic acid sequence, or translocate a nucleic acid sequence. In this system, Cre recombinase may recombine a pair of lox nucleic acid sequences. Lox sites comprise 34 nucleotides, with the first and last 13 nucleotides (arms) being palindromic. During recombination, Cre recombinase protein binds to two lox sites on different nucleic acids and cleaves at the lox sites. The cleaved nucleic acids are spliced together (reciprocally translocated) and recombination is complete. In another aspect, a lox site provided herein is a loxP, lox 2272, loxN, lox 511, lox 5171, lox71, ox66, M2, M3, M7, or M11 site.

ZFNs are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The DNA-binding domain of a ZFN may typically be composed of 3-4 (or more) zinc-fingers. The amino acids at positions −1, +2, +3, and +6 relative to the start of the zinc finger α-helix, which contribute to site-specific binding to the target site, can be changed and customized to fit specific target sequences. The other amino acids may form a consensus backbone to generate ZFNs with different sequence specificities. Methods and rules for designing ZFNs for targeting and binding to specific target sequences are known in the art. See, e.g., US Patent App. Nos. 2005/0064474, 2009/0117617, and 2012/0142062, the contents and disclosures of which are incorporated herein by reference. The FokI nuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site.

Without being limited by any scientific theory, because the DNA-binding specificities of zinc finger domains can be re-engineered using one of various methods, customized ZFNs can theoretically be constructed to target nearly any target sequence (e.g., at or near a gene in a plant genome). Publicly available methods for engineering zinc finger domains include Context-dependent Assembly (CoDA), Oligomerized Pool Engineering (OPEN), and Modular Assembly. In an aspect, a method and/or composition provided herein comprises one or more, two or more, three or more, four or more, or five or more ZFNs. In another aspect, a ZFN provided herein can generate a targeted DSB or nick.

Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). According to some embodiments, a meganuclease may comprise a scaffold or base enzyme selected from the group consisting of I-CreI, I-CeuI, I-MsoI, I-SceI, I-AniI, and I-DmoI. The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity. Thus, a meganuclease may be selected or engineered to bind to a genomic target sequence in a plant, such as at or near the genomic locus of a gene. In another aspect, a meganuclease provided herein can generate a targeted DSB.

TALENs are artificial restriction enzymes generated by fusing the transcription activator-like effector (TALE) DNA binding domain to a nuclease domain (e.g., FokI). When each member of a TALEN pair binds to the DNA sites flanking a target site, the FokI monomers dimerize and cause a double-stranded DNA break at the target site. Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity.

TALENs are artificial restriction enzymes generated by fusing the transcription activator-like effector (TALE) DNA binding domain to a nuclease domain. In some aspects, the nuclease is selected from a group consisting of PvuII, MutH, TevI, FokI, AlwI, MlyI, SbfI, SdaI, StsI, CleDORF, Clo051, and Pept071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN also refers to one or both members of a pair of TALENs that work together to cleave DNA at the same site.

Transcription activator-like effectors (TALEs) can be engineered to bind practically any DNA sequence, such as at or near the genomic locus of a gene in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (see Yank et al. 2013. PLoS One. 8: e82539). MutH can introduce strand-specific nicks in DNA (see Gabsalilow et al. 2013. Nucleic Acids Research. 41: e83). TevI introduces double-stranded breaks in DNA at targeted sites (see Beurdeley et al., 2013. Nature Communications. 4: 1762).

The relationship between amino acid sequence and DNA recognition of the TALE binding domain allows for designable proteins. Software programs such as DNA Works can be used to design TALE constructs. Other methods of designing TALE constructs are known to those of skill in the art. See Doyle et al., Nucleic Acids Research (2012) 40: W117-122; Cermak et al., Nucleic Acids Research (2011). 39:e82; and tale-nt.cac.cornell.edu/about. In another aspect, a TALEN provided herein can generate a targeted DSB.

According to some embodiments, a donor template may also be present in the surrounding medium, etc., and introduced, transferred or delivered (i.e., transferred) to the recipient cell to serve as a template for the desired edit generated following the introduction of a double-stranded break (DSB) or nick in the recipient cell genome by the site-specific nuclease. Alternatively, a donor template may be present in or expressed by the recipient cell. Similarly, for RNA-guided nuclease, a transcribable DNA sequence or transgene expressing a guide RNA (gRNA) may also be present in the surrounding medium, etc., and introduced, transferred or delivered (i.e., transferred) to the recipient cell to serve as a guide RNA for the RNA-guided nuclease to direct the RNA-guided nuclease to make a double-stranded break (DSB) or nick at the desired locus or target site in the recipient cell genome. Alternatively, a guide RNA (gRNA) may be present in or expressed by the recipient cell. According to further embodiments, (i) a site-specific nuclease, a guide RNA and a donor template may all be present in the surrounding medium, etc., and become introduced, transferred or delivered to a recipient cell, or (ii) a site-specific nuclease and/or a guide RNA may be present in the surrounding medium, etc., and become introduced, transferred or delivered to a recipient cell, and a donor template may be optionally present in or expressed by the recipient cell, or (iii) a site-specific nuclease and/or a donor template may be present in the surrounding medium, etc., and become introduced, transferred or delivered to a recipient cell, and a guide RNA may be optionally present in or expressed by the recipient cell, or (iv) a guide RNA and/or a donor template may be present in the surrounding medium, etc., and become introduced, transferred or delivered to a recipient cell, and a site-specific nuclease may be present in or expressed by the recipient cell, in each case (i), (ii), (iii) or (iv) to make a double-stranded break (DSB) or nick at the desired locus or target site in the recipient cell genome to give rise to a templated or non-templated edit or mutation a the desired location in the genome of the recipient cell.

Any site or locus within the genome of a plant may potentially be chosen for making a genomic edit (or gene edit) or site-directed integration of a transgene, construct or transcribable DNA sequence. For genome editing and site-directed integration, a double-strand break (DSB) or nick may first be made at a selected genomic locus with a site-specific nuclease, such as, for example, a zinc-finger nuclease (ZFN), an engineered or native meganuclease, a TALE-endonuclease, or an RNA-guided endonuclease (e.g., Cas9 or Cpf1). Any method known in the art for site-directed integration may be used. In the presence of a donor template molecule with an insertion sequence, the DSB or nick can be repaired by homologous recombination between homology arm(s) of the donor template and the plant genome, or by non-homologous end joining (NHEJ), resulting in site-directed integration of the insertion sequence into the plant genome to create the targeted insertion event at the site of the DSB or nick. Thus, site-specific insertion or integration of a transgene, transcribable DNA sequence, construct or sequence may be achieved if the transgene, transcribable DNA sequence, construct or sequence is located in the insertion sequence of the donor template.

The introduction of a DSB or nick may also be used to introduce targeted mutations in the genome of a plant. According to this approach, mutations, such as deletions, insertions, inversions and/or substitutions may be introduced at a target site via imperfect repair of the DSB or nick to produce a knock-out or knock-down of a gene. Such mutations may be generated by imperfect repair of the targeted locus even without the use of a donor template molecule. A “knock-out” of a gene may be achieved by inducing a DSB or nick at or near the endogenous locus of the gene that results in non-expression of the protein or expression of a non-functional protein, whereas a “knock-down” of a gene may be achieved in a similar manner by inducing a DSB or nick at or near the endogenous locus of the gene that is repaired imperfectly at a site that does not affect the coding sequence of the gene in a manner that would eliminate the function of the encoded protein. For example, the site of the DSB or nick within the endogenous locus may be in the upstream or 5′ region of the gene (e.g., a promoter and/or enhancer sequence) to affect or reduce its level of expression. Similarly, such targeted knock-out or knock-down mutations of a gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, inversions and/or substitutions, relative to the targeted genomic sequence at or near the site of the DSB or nick. For example, targeted knock-out mutations of a gene may be achieved by deleting or inverting at least a portion of the gene or by introducing a frame shift or premature stop codon into the coding sequence of the gene. A deletion of a portion of a gene may also be introduced by generating DSBs or nicks at two target sites and causing a deletion of the intervening target region flanked by the target sites.

As used herein, a “donor molecule”, “donor template”, or “donor template molecule” (collectively a “donor template”), which may be a recombinant polynucleotide, DNA or RNA donor template, is defined as a nucleic acid molecule having a nucleic acid template or insertion sequence for site-directed, targeted insertion or recombination into the genome of a plant cell via repair of a nick or double-stranded DNA break in the genome of a plant cell. For example, a “donor template” may be used for site-directed integration of a transgene or suppression construct, or as a template to introduce a mutation, such as an insertion, deletion, substitution, etc., into a target site within the genome of a plant. A targeted genome editing technique provided herein may comprise the use of one or more, two or more, three or more, four or more, or five or more donor molecules or templates. A “donor template” may be a single-stranded or double-stranded DNA or RNA molecule or plasmid. An “insertion sequence” of a donor template is a sequence designed for targeted insertion into the genome of a plant cell, which may be of any suitable length. For example, the insertion sequence of a donor template may be between 2 and 50,000, between 2 and 10,000, between 2 and 5000, between 2 and 1000, between 2 and 500, between 2 and 250, between 2 and 100, between 2 and 50, between 2 and 30, between 15 and 50, between 15 and 100, between 15 and 500, between 15 and 1000, between 15 and 5000, between 18 and 30, between 18 and 26, between 20 and 26, between 20 and 50, between 20 and 100, between 20 and 250, between 20 and 500, between 20 and 1000, between 20 and 5000, between 20 and 10,000, between 50 and 250, between 50 and 500, between 50 and 1000, between 50 and 5000, between 50 and 10,000, between 100 and 250, between 100 and 500, between 100 and 1000, between 100 and 5000, between 100 and 10,000, between 250 and 500, between 250 and 1000, between 250 and 5000, or between 250 and 10,000 nucleotides or base pairs in length. A donor template may also have at least one homology sequence or homology arm, such as two homology arms, to direct the integration of a mutation or insertion sequence into a target site within the genome of a plant via homologous recombination, wherein the homology sequence or homology arm(s) are identical or complementary, or have a percent identity or percent complementarity, to a sequence at or near the target site within the genome of the plant. When a donor template comprises homology arm(s) and an insertion sequence, the homology arm(s) will flank or surround the insertion sequence of the donor template.

An insertion sequence of a donor template may comprise one or more genes or sequences that each encode a transcribed non-coding RNA or mRNA sequence and/or a translated protein sequence. A transcribed sequence or gene of a donor template may encode a protein or a non-coding RNA molecule. An insertion sequence of a donor template may comprise a polynucleotide sequence that does not comprise a functional gene or an entire gene sequence (e.g., the donor template may simply comprise regulatory sequences, such as a promoter sequence, or only a portion of a gene or coding sequence), or may not contain any identifiable gene expression elements or any actively transcribed gene sequence. Further, the donor template may be linear or circular, and may be single-stranded or double-stranded. A donor template may be delivered to a cell as a RNA molecule expressed from a transgene. A donor template may also be delivered to the cell as a naked nucleic acid (e.g., via particle bombardment), as a complex with one or more delivery agents (e.g., liposomes, proteins, poloxamers, T-strand encapsulated with proteins, etc.), or contained in a bacterial or viral delivery vehicle, such as, for example, Agrobacterium tumefaciens or a geminivirus, respectively. An insertion sequence of a donor template provided herein may comprise a transcribable DNA sequence that may be transcribed into an RNA molecule, which may be non-coding and may or may not be operably linked to a promoter and/or other regulatory sequence.

According to some embodiments, a donor template may not comprise an insertion sequence, and instead comprise one or more homology sequences that include(s) one or more mutations, such as an insertion, deletion, substitution, etc., relative to the genomic sequence at a target site within the genome of a plant, such as at or near a gene within the genome of a plant. Alternatively, a donor template may comprise an insertion sequence that does not comprise a coding or transcribable DNA sequence, wherein the insertion sequence is used to introduce one or more mutations into a target site within the genome of a plant, such as at or near a gene within the genome of a plant.

A donor template provided herein may comprise at least one, at least two, at least three, at least four, at least five, at least six, at least seven, at least eight, at least nine, or at least ten genes or transcribable DNA sequences. Alternatively, a donor template may comprise no genes. Without being limiting, a gene or transcribable DNA sequence of a donor template may include, for example, an insecticidal resistance gene, an herbicide tolerance gene, a nitrogen use efficiency gene, a water use efficiency gene, a yield enhancing gene, a nutritional quality gene, a DNA binding gene, a selectable marker gene, an RNAi or suppression construct, a site-specific genome modification enzyme gene, a single guide RNA of a CRISPR/Cas9 system, a geminivirus-based expression cassette, or a plant viral expression vector system. According to other embodiments, an insertion sequence of a donor template may comprise a protein encoding sequence or a transcribable DNA sequence that encodes a non-coding RNA molecule, which may target an endogenous gene for suppression. A donor template may comprise a promoter, such as a constitutive promoter, a tissue-specific or tissue-preferred promoter, a developmental stage promoter, or an inducible promoter. A donor template may comprise a leader, enhancer, promoter, transcriptional start site, 5′-UTR, one or more exon(s), one or more intron(s), transcriptional termination site, region or sequence, 3′-UTR, and/or polyadenylation signal. The leader, enhancer, and/or promoter may be operably linked to a gene or transcribable DNA sequence encoding a non-coding RNA, a guide RNA, an mRNA and/or protein.

According to present embodiments, a portion of a recombinant donor template polynucleotide molecule (i.e., an insertion sequence) may be inserted or integrated at a desired site or locus within the plant genome. The insertion sequence of the donor template may comprise a transgene or construct, such as a transgene or transcribable DNA sequence encoding a non-coding RNA molecule that targets an endogenous gene for suppression. The donor template may also have one or two homology arms flanking the insertion sequence to promote the targeted insertion event through homologous recombination and/or homology-directed repair. Each homology arm may be at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence within the genome of a plant cell. Thus, a plant cell may comprise a recombinant DNA molecule encoding a donor template for site-directed or targeted integration of a transgene or construct, such as a transgene or transcribable DNA sequence encoding a non-coding RNA molecule that targets an endogenous gene for suppression, into the genome of a plant.

As used herein, a “target site” for genome editing or site-directed integration refers to the location of a polynucleotide sequence within a plant genome that is bound and cleaved by a site-specific nuclease introducing a double stranded break (or single-stranded nick) into the nucleic acid backbone of the polynucleotide sequence and/or its complementary DNA strand. A target site may comprise at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for a RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further below). A non-coding guide RNA provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a plant genome that is bound and cleaved by another site-specific nuclease that may not be guided by a non-coding RNA molecule, such as a meganuclease, zinc finger nuclease (ZFN), or a transcription activator-like effector nuclease (TALEN), to introduce a double stranded break (or single-stranded nick) into the polynucleotide sequence and/or its complementary DNA strand.

As used herein, a “target region” or a “targeted region” refers to a polynucleotide sequence or region that is flanked by two or more target sites. Without being limiting, in some embodiments a target region may be subjected to a mutation, deletion, insertion or inversion. As used herein, “flanked” when used to describe a target region of a polynucleotide sequence or molecule, refers to two or more target sites of the polynucleotide sequence or molecule surrounding the target region, with one target site on each side of the target region.

### EXAMPLES

The following examples are included to demonstrate certain embodiments of the present disclosure. It should be appreciated by those of skill in the art that these examples that follow represent techniques and approaches that may be used in the practice of present methods and embodiments. However, those of skill in the art should, in light of the present disclosure, appreciate that many modifications, changes and substitutions may be made to the specific embodiments disclosed herein to obtain similar results without departing from the spirit and scope of the present disclosure.

### Example 1: Delivery of TAT-Cre Protein into Wounded Corn Callus Cells

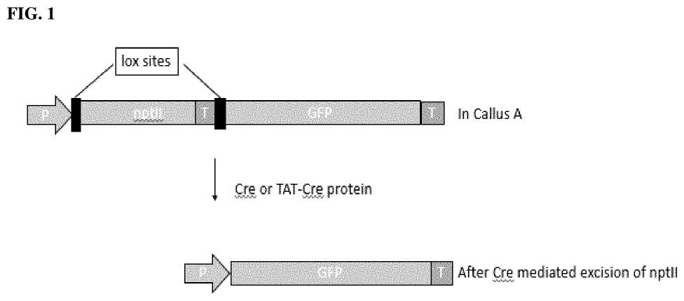

Transgenic corn line A was created having a recombinant DNA construct in its nuclear genome including, in the 5′ to 3′ direction, an enhanced CaMV 35S promoter with an HSP70 intron in the 5′ untranslated region, a nptII selectable marker gene cassette flanked by two lox sites, followed by a green fluorescent protein (GFP) encoding gene (see, e.g., Zhang et al., Theor. Appl. Gen. 107(7):1157-1168; 2003). In this arrangement, GFP is not functionally expressed due to the intervening nptII gene between the 35S promoter and the GFP coding sequence. However, in the presence of Cre recombinase enzyme, the nptII gene is excised due to the flanking lox sites, which results in high levels of GFP expression that can be visualized in most tissues by bringing the 35S promoter and the GFP coding sequence together (FIG. 1). Thus, the GFP construct inserted into the genome of the corn line A can be used as a reporter for the presence and activity of Cre recombinase in one or more cells of the transgenic corn line. Embryogenic callus cells were generated from immature embryos of transgenic corn line A using methods known in the art (see, e.g., Sidorov and Duncan, Methods in Molecular Biology, Vol. 526, Transgenic Maize Methods and Protocols, Humana Press (2009)). About 1.5 grams of callus cells from transgenic corn lines A were chopped into fine pieces, packed into clumps, and grown on Medium 1074. The composition of Medium 1074 is shown in Table 1.

TABLE 1
Composition of Medium 1074

| Ingredient | Ingredient Description | Amount | |
|---|---|---|---|
| MS_BASAL_SALT | MS Basal Salts | 2.165 | g |
| MP00266 | MS Vitamins (100X) | 5.000 | mL |
| SUCROSE | Sucrose | 20.000 | g |
| TC_WATER_TO_VOLUME | Bring to volume with | 1000.000 | mL |
| | TC water | | |
| PH_WITH_KOH_TO | pH with KOH to | 5.800 | |
| GELZAN_CM | Gelzan CM | 3.000 | g |
| AUTOCLAVE | Autoclave | | |
| MP00255 | IBA (1 mg/ml) | 0.750 | mL |
| MP00161 | NAA (1 mg/mL) | 0.500 | mL |

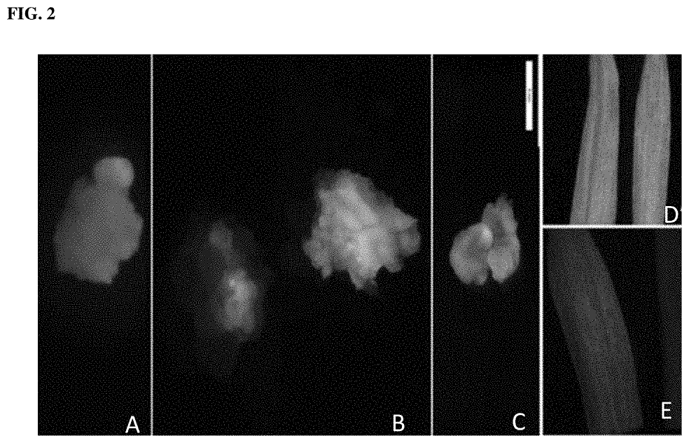

TAT-Cre recombinase purchased from Millipore is a recombinant cell-permeant fusion protein consisting of a basic protein translocation peptide derived from HIV-TAT (TAT), a nuclear localization sequence (NLS), the Cre protein, and an N-terminal histidine tag (H6) for efficient purification of the protein from E. coli. (http://www.emdmillipore.com/US/en/product/TAT-CRE-Recombinase, MMNF-SCR508). A solution of TAT-Cre protein was made at a concentration of 15.6 μg or 31.2 μg of the TAT-Cre protein in 200 μl of PBS buffer. To test for the ability to transfer and deliver the TAT-Cre recombinase protein into callus cells, several experiments were carried out with callus cells from corn line A having the GFP reporter construct. In each treatment, 2-3 ml of packed callus cells (after chopping) prepared as described above were used without washing and spread directly on Medium 1074. After 3 weeks of culture, the first GFP positive callus pieces were identified (FIGS. 2A-C). Four GFP-positive calli were isolated and sub-cultured on the same medium. Three of the GFP-positive callus lines were regenerable and also gave rise to shoots. These regenerated plants were also found to have GFP expression in leaves (FIGS. 2D and 2E). The plants were transferred to rooting Medium 1796 (Table 2) and subsequently transferred to the greenhouse.

TABLE 2
Composition of Medium 1796

| Ingredient | Ingredient Description | Amount | |
|---|---|---|---|
| MS_BASAL_SALT | MS Basal Salts | 4.330 | g |
| MP00266 | MS Vitamins (100X) | 10.000 | mL |
| MP00033 | Thiamine HCL | 1.000 | mL |
| | (0.5 mg/ml) | | |
| 2_4_D_1 MG_ML | 2,4-D (1 mg/ml) | 0.500 | mL |
| SUCROSE | Sucrose | 30.000 | g |
| PROLINE | Proline | 1.380 | g |
| CASAMINO_ACIDS | Casamino Acids | 0.500 | g |
| TC_WATER_TO_VOLUME | Bring to volume with | 1000.000 | mL |
| | TC water | | |
| PH_WITH_KOH_TO | pH with KOH to | 5.800 | |
| GELZAN_CM | Gelzan CM | 3.000 | g |
| AUTOCLAVE | Autoclave | | |
| MP00158 | Picloram (1 mg/mL) | 2.200 | mL |
| MP00279 | Silver Nitrate | 1.700 | mL |
| | (2 mg/ml) | | |

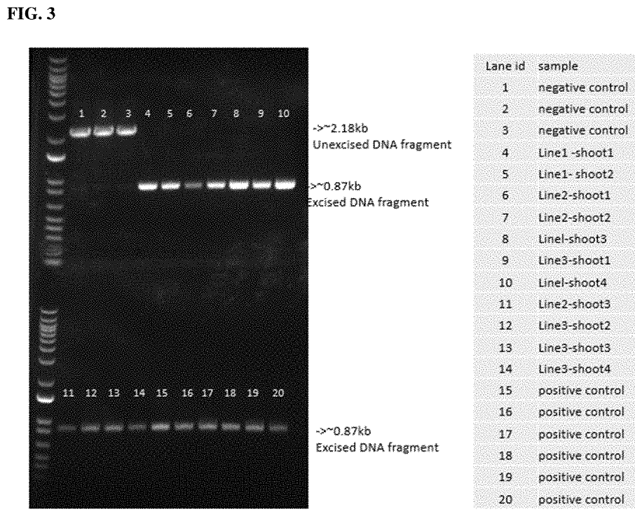

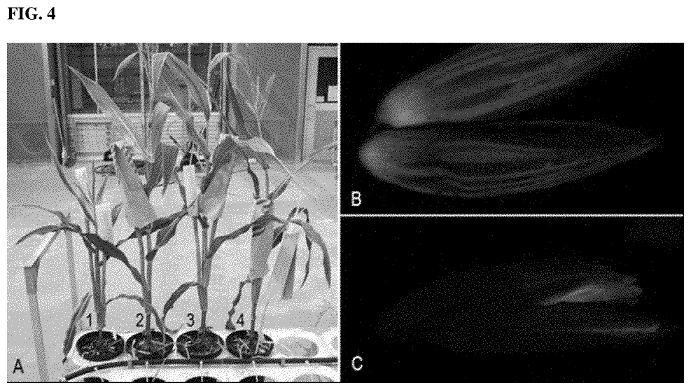

Samples taken from shoot tissue of regenerated GFP-positive and negative plants (FIG. 4A) were subjected to PCR using primers designed to amplify the GFP-reporter construct. Genomic DNA samples taken from plants 1-3 regenerated from GFP-positive callus produced a fragment having the predicted size with the Cre-lox excision (see FIG. 3; shoot samples from GFP positive plants 1-3, respectively, had the excised band, whereas shoot samples from the GFP negative control plant 4 had the predicted longer unexcised fragment size). FIG. 3 further shows the excised band from positive control samples with the excised construct. GFP expression in tassel spikelet of the GFP positive plant 2 was also visualized under a blue light (FIG. 4B), as compared to no GFP expression in a tassel spikelet from the GFP-negative control plant 4 (FIG. 4C).

Genomic DNA was isolated from leaf tissue using the CTAB method known in the art. PCR reactions were carried out using PrimeStar GXL Polymerase (TAKARA) and a set of primers hybridizing upstream and downstream of the GFP reporter construct, and the PCR products from these reactions were resolved in 1% agarose gel (FIG. 3). Cre-excision of the nptII gene cassette was confirmed by the presence of a ˜0.97 kb band for the excised DNA fragment, as compared to a ˜2.18 kb band for unexcised DNA fragment. Sequencing through the expected recombination junction also confirmed the excision of the nptII gene.

### Example 2: Delivery of Cre Protein into Wounded Corn Callus Cells

Embryogenic callus cells were generated from immature embryos of transgenic corn line A as described in Example 1 above. 3-4 grams of callus was blended at a high speed for 9-10 seconds in a medium containing 50% concentration of medium 4278 (Table 3) and 0.3M mannitol to obtain a fine suspension of callus pieces sized 1-2 mm. After blending, the callus suspension was poured through the sieves, washed with the medium used for blending, and blotted onto the filter paper.

TABLE 3
Composition of Medium 4278

| Ingredient | Ingredient Description | Amount | |
|---|---|---|---|
| MP00927 | FN-lite macro stock (10X) | 100.000 | mL |
| MS_MICRONUTRIENT | MS Micronutrients | 100.000 | mL |
| GAMBORGS_B5_500X | Gamborgs B5 500X | 2.000 | mL |
| SUCROSE | Sucrose | 30.000 | g |
| ASPARAGINE_MONOHYD | Asparagine monohydrate | 1.000 | g |
| TC_WATER_TO_VOLUME | Bring to volume with TC water | 1000.000 | mL |
| PH_WITH_KOH_TO | pH with KOH to | 5.700 | |
| FILTER_STERILIZE_022MICRON | Filter sterilize with 0.22 micron unit | | |

Recombinant Cre protein was made using publicly known methods (J Mol Biol.; 313(1):49-69; 2001). The recombinant Cre protein was put in a buffer of 25 mM Tris, pH 8.0 and 300 mM NaCl at a concentration of 4.8 mg/ml and filtered through a 0.2 micron filter to be sterilized. 1 ml of this recombinant Cre solution was added into 3 ml of the blended callus suspension. Two treatments were conducted in this experiment. In one treatment, the recombinant Cre protein solution was added to the blended callus suspension and treated with 1 ml of PEG solution (Table 4).

TABLE 4
Composition of PEG solution

| | Stock | for 10 ml | |
|---|---|---|---|
| PEG 40000 (Mallinckrodt Baker, Inc.) | n/a | 4.0 | g |
| Water | n/a | 2.0 | ml |
| Mannitol | 0.8M | 3.0 | ml |
| Ca(NO₃)₂ × 4H₂0 | 1M | 1.0 | ml |

Callus was carefully mixed with protein/PEG and left on the plate for 10 minutes, and W5 medium (Table 5) was slowly added and then mostly removed to wash off the Cre/PEG

### Solution

TABLE 5
Composition of W5 solution

| | Stock | for 500 ml |
|---|---|---|
| 154 mM NaCl | 5M | 15.4 ml |
| 125 mM CaCl₂ | 1M | 62.5 ml |
| 5 mM KCl | 1M | 2.5 ml |
| 2 mM MES (pH 5.7) | 0.2M | 5.0 ml |
| Water | n/a | 414.6 ml |

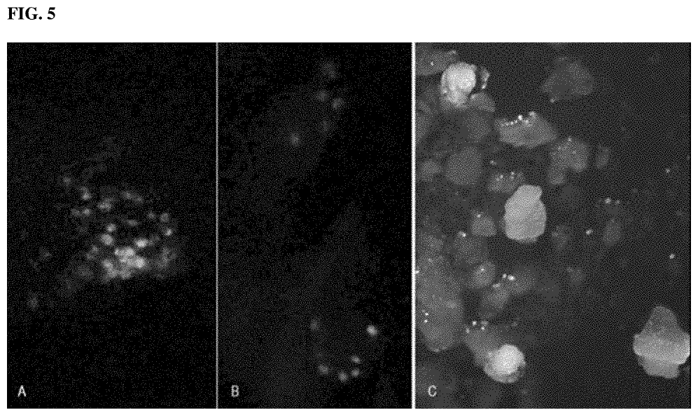

Clumps of cells were resuspended in a small volume of fresh W5 medium and transferred to a plate containing medium 1074. Cell clumps were uniformly spread over the plate, and liquid was removed with a fine pipette. Plates were incubated in a Percival container at 28° C. In a second treatment, the recombinant Cre protein solution was added to the blended callus suspension directly (i.e., without PEG). In both treatments, the plates of cells containing medium 1074 were cultured for at least three days and then analyzed for GFP expression. In these experiments, GFP expression was only observed in plates containing the blended callus suspension that had been treated with PEG after three days of culturing (FIGS. 5A and B), and after six days of culturing (FIG. 5C). No GFP expression was found in plates where callus with Cre was not treated with PEG.

### Example 3: Delivery of RNP into Wounded Corn Callus Cells

The ability to deliver a Cre recombinase enzyme to wounded callus cells suggests that other proteins, ribonucleoproteins and nucleases could also be added to cells by this method. Embryogenic callus cells are generated from corn immature embryos as described in Example 1 above, and a wounded callus suspension is generated as described in Example 2 above. The blended callus suspension is washed and dried as described above. The PEG solution can be added as described above when a RNP complex solution described below is mixed with wounded callus cells.

To generate a guide RNA-Cas9 ribonucleoprotein (RNP) complex, 20.6 μg of Cas9 protein and 8.6 μg of gRNA are mixed to a Cas9 to gRNA molar ratio of 1:2 in 1×NEB buffer 3 (100 mM NaCl, 50 mM Tris-HCl, 10 mM MgCl₂, 1 mM DTT, pH 7.9 at 25° C.) and 1 μl of RNase inhibitor (RiboLock; Thermo Fisher Scientific) to a total volume of 30 μl and incubated at room temperature for at least 1.5 min. Optionally for co-delivery, an aadA PCR product is added to the premix. To generate a guide RNA-Cpf1 RNP complex, Cpf1 protein at the concentration of 6.6 mg/ml (44.7 uM) is mixed with gRNA to create a Cpf1 to gRNA molar ratio of 1:5.

Wounded callus cells are incubated with the RNP complex solution for a predetermined period of time. After incubation, the callus suspension is plated onto medium 1074 and cultured as described above to generate plants. The plants are then transferred into rooting medium 1796 and subsequently transferred to the greenhouse. Parts of the plants are harvested for molecular and phenotypic analysis confirming gene or genome edit by the delivered RNP.

While the present invention has been disclosed with reference to certain embodiments, it will be apparent that modifications and variations are possible without departing from the spirit and scope of the present invention as described herein and as provided by the appended claims. Furthermore, it should be appreciated that all examples in the present disclosure, while illustrating embodiments of the invention, are provided as non-limiting examples and are, therefore, not to be taken as limiting the various aspects so illustrated. The present invention is intended to have the full scope defined by the present disclosure, the language of the following claims, and any equivalents thereof. Accordingly, the examples, drawings and detailed description are to be regarded as illustrative and not as restrictive.

1. A method for transfer of a biomolecule into a cell comprising:
a) mixing a recipient plant cell culture comprising at least one recipient cell with a medium comprising at least one biomolecule to obtain a mixed cell culture comprising the recipient cell; and
b) wounding the recipient cell of the mixed cell culture to produce at least one product cell into which transfer of the biomolecule has occurred following said mixing and/or wounding.

2-27. (canceled)

28. A method for transfer of a biomolecule into a cell comprising:
a) wounding a recipient cell of a recipient plant cell culture; and
b) mixing the recipient cell culture with a medium comprising at least one biomolecule to obtain a mixed cell culture comprising the recipient cell and to produce at least one product cell into which transfer of the biomolecule has occurred following said wounding and/or mixing.

29-48. (canceled)

49. A method for editing a plant cell comprising:
a) mixing a recipient plant cell culture comprising a recipient cell with a medium comprising at least one biomolecule to obtain a mixed cell culture comprising the recipient cell, wherein the biomolecule comprises a site-specific nuclease or a recombinant DNA molecule comprising a sequence encoding a site-specific nuclease operably linked to a first promoter; and
b) wounding the recipient cell of the mixed cell culture to produce at least one edited product cell having an edit or mutation introduced in its genome by the site-specific nuclease.

50. A method for editing a plant cell comprising:
a) wounding a recipient cell of a recipient plant cell culture; and
b) mixing the recipient plant cell culture with a medium comprising at least one biomolecule to obtain a mixed cell culture comprising the recipient cell, wherein the biomolecule comprises a site-specific nuclease or a recombinant DNA molecule comprising a sequence encoding a site-specific nuclease operably linked to a first promoter, to produce at least one edited product cell having an edit or mutation introduced in its genome by the site-specific nuclease.

51. The method of claim 50, further comprising:
screening or selecting for the at least one edited product cell, or a progeny cell thereof, or a plant developed or regenerated from the at least one edited product cell, or a progeny cell thereof, having the edit or mutation;
adding an osmoticum to the recipient plant cell culture, medium or mixed cell culture prior to, during or after step a) or step b); or
regenerating a plant from the mixed cell culture and/or the at least one edited product cell, or at least one progeny cell thereof.

52. The method of claim 50, wherein:
the recipient plant cell culture, medium or mixed cell culture further comprises an osmoticum;
the recipient plant cell culture is a callus culture or cell suspension culture;
cells of the recipient plant cell culture are dicot plant cells or monocot plant cells;
the first promoter operably linked to the sequence encoding a site-specific nuclease is a constitutive promoter, a tissue-specific or tissue-preferred promoter, a developmental stage promoter, or an inducible promoter;
the site-specific nuclease is a zinc-finger nuclease (ZFN), a meganuclease, an RNA-guided endonuclease, a TALE-endonuclease (TALEN), a recombinase, or a transposase;
the medium further comprises a first recombinant DNA construct comprising a first transcribable DNA sequence encoding a guide RNA molecule operably linked to a promoter;
the medium further comprises a donor template molecule or a second recombinant DNA construct comprising a second transcribable DNA sequence encoding a donor template molecule operably linked to a promoter;
one or more cells of the recipient plant cell culture comprise a recombinant DNA construct comprising a first transcribable DNA sequence encoding a guide RNA molecule operably linked to a promoter; or
the recipient cell of the recipient plant cell culture comprises a donor template molecule or a recombinant DNA construct comprising a second transcribable DNA sequence encoding a donor template molecule operably linked to a promoter.

53. (canceled)

54. The method of claim 52, wherein the osmoticum comprises:
(a) polyethylene glycol (PEG); or
(b) a sugar or sugar alcohol.

55. The method of claim 51, wherein:
the plant developed or regenerated from the at least one edited product cell, or a progeny cell thereof, is screened or selected based on a trait or phenotype produced by the edit or mutation and present in the developed or regenerated plant, or a progeny plant, plant part or seed thereof; or
the at least one edited product cell, or a progeny cell thereof, or the plant developed or regenerated from the at least one edited product cell, or a progeny cell thereof, are screened or selected based on a molecular assay.

56-58. (canceled)

59. The method of claim 51, further comprising
d) regenerating a plant from the mixed cell culture and/or the at least one edited product cell, or at least one progeny cell thereof.

60. (canceled)

61. The method of claim 52, wherein:
the dicot plant cells are selected from the group consisting of tobacco, tomato, soybean, canola, and cotton cells;
the monocot plant cells are selected from the group consisting of corn, rice, wheat, barley, and sorghum cells;
the site-specific nuclease is an RNA-guided nuclease;
the promoter operably linked to the first transcribable DNA sequence is a constitutive promoter, a tissue-specific or tissue-preferred promoter, a developmental stage promoter, or an inducible promoter;
the donor template molecule comprises a transgene comprising a coding sequence or transcribable DNA sequence operably linked to a plant-expressible promoter; or
the promoter operably linked to the second transcribable DNA sequence is a constitutive promoter, a tissue-specific or tissue-preferred promoter, a developmental stage promoter, or an inducible promoter.

62-72. (canceled)

73. An edited product cell produced by the method of claim 50.

74. The edited product cell of claim 73, wherein the plant cell is a dicot plant cell or a monocot plant cell.

75. A plant regenerated or developed from the edited product cell produced by the method of claim 50, or a progeny cell thereof.

76. The regenerated plant of claim 98, wherein the plant is a dicot or monocot plant.

77. A seed, progeny plant, or progeny seed of the plant of claim 76.

78. A wounded mixed cell culture produced by the method of claim 50.
